(19) United States
(12) Patent Application Publication
(10) Pub. No.: US 2005/0228218 A1
(43) Pub. Date: Oct. 13, 2005

(54) Double anchor strapless dildo

(76) Inventors: Steven S. Skidmore, Pfluegerville, TX (US); Carolyn F. Skidmore, Pfluegerville, TX (US)

Correspondence Address: Derek R. Van Gilder, 916 Main St., Bastrop, TX, 78602, US

(21) Appl. No.: 10/822,270

(22) Filed: Apr. 9, 2004

(51) Int. Cl. A61F 5/00

(52) U.S. Cl. 600/38

(57) ABSTRACT

A double-anchor strapless dildo has an insertion shaft (1) formed for insertion in a vagina of a first female by a second female to whom the insertion shaft is double-anchored with an anal anchor (4) and a vaginal anchor (5) on an attachment base (2). The insertion shaft is formed and optionally surfaced for desired contact with erogenous areas of the vagina of the first female. The vaginal anchor is formed and optionally surfaced for desired contact with erogenous areas of the vagina of the second female. For combining attachment rigidity and contact with erogenous areas of the second female, the attachment base has a vaginal-anchor hook (11) formed for engaging bony structure of the symphysis pubis and for engaging erogenous entry walls of the vagina of the second female while being double-anchored to the second female with the anal anchor and the vaginal anchor.

### BACKGROUND OF THE INVENTION

1. Field of the Invention

This invention relates to dildos for vaginal insertion.

2. Relation to prior art.

There are a wide variety of known sexual-gratification items that include dildos for vaginal insertion. There are none known to be strapless for female-to-female use as taught by this invention.

### SUMMARY OF THE INVENTION

Objects of patentable novelty and utility of this invention are to provide a double-anchor strapless dildo having a vaginal anchor and a rectal anchor for reliably rigid and gratifying insertion into a first female to support a penal-like dildo for being gratifyingly inserted into the vagina of a second female in a manner taught by this invention.

### BRIEF DESCRIPTION OF DRAWINGS

This invention is described by appended claims in relation to description of a preferred embodiment with reference to the following drawing which is explained briefly as follows:

FIG. 1 is a side elevation view.

### DESCRIPTION OF PREFERRED EMBODIMENT

A description of the preferred embodiment of this invention follows a list of numbered terms which designate its features with the same numbers on the drawings and in parentheses throughout the description and throughout the patent claims.
1. Insertion shaft
2. Attachment base
3. Insertion end
4. Anal anchor
5. Vaginal anchor
6. Circular point
7. Circular base
8. Anal-anchor protrusion
9. Vaginal-anchor shaft
10. Vaginal-anchor head
11. Vaginal-anchor hook
12. Insertion-shaft head
13. Base end
14. Terminal end

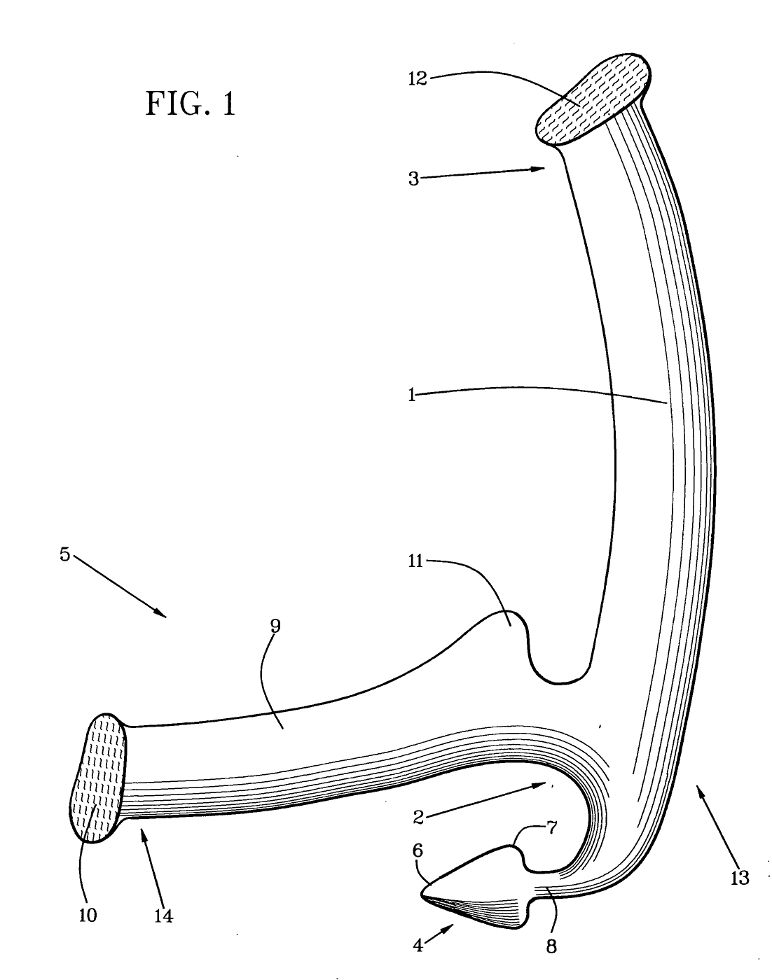

Referring to the drawing, FIG. 1, a double-anchor strapless dildo has an insertion shaft (1) extended intermediate an attachment base (2) and an insertion end (3). The insertion shaft (1) has length and breadth for gratifying insertion into a vagina of a first female. The attachment base (2) has an anal anchor (4) and a vaginal anchor (5). The vaginal anchor (5) has length and breadth for gratifying insertion into a vagina of a second female. The anal anchor (4) and the vaginal anchor (5) are extended from an attachment side of the attachment base (2) with the vaginal anchor (5) being intermediate the anal anchor (4) and the insertion shaft (1).

The anal anchor (4) has breadth for ease of insertion into an anus of the second female and form for deterring escape from the anus of the second female.

The vaginal anchor (5) has breadth for ease of insertion into the vagina of the second female and for deterring escape from the vagina of the second female.

The anal anchor (4) includes a conically circular point (6) having a circular base (7) extended radially outward from an anal-anchor protrusion (8) extended from the attachment side of the attachment base (2). The conically circular point (6) is blunted and elongated predeterminedly for ease of entry into and gentle spread of the anal orifice of the second female. The conically circular base (7) includes a rounded edge for hooking bluntly onto an inside of an entry wall of the anal orifice of the second female. The anal-anchor protrusion (8) can include a length that is proximate a thickness of the entry wall of the anal orifice of the second female.

The vaginal anchor (5) can include a vaginal-anchor shaft (9) having length, breadth and shape for gratifying insertion into the vagina of the second female.

The vaginal-anchor shaft (9) preferably includes a predetermined vaginal-anchor head (10) formed and structured proximate a terminal end (14) for stimulating entry into and linear positioning within the vagina of the second female.

The vaginal anchor (5) can have a vaginal-anchor hook (11) proximate a base end of the vaginal-anchor shaft (9). The vaginal-anchor hook (11) is formed and positioned for engaging forward walls of the vagina and for engaging bony structure of the symphysis pubis of the second female.

The insertion shaft (1) can include at least one predetermined insertion-shaft head (12) formed and positioned on the insertion shaft (1) for engaging erogenous zones of the vagina of the first female.

The insertion shaft (1), the anal anchor (4) and the vaginal anchor (5) are preferably rigid and washable.

The insertion shaft (1) is structured for deterrence of lateral pivot by double anchoring with the anal anchor (4) and the vaginal anchor (5).

The attachment base (2) is formed for deterring unwantedly loose linear travel of the insertion shaft (1) in the vagina of the first female by double anchoring with the anal anchor (4) being buttressed by aft bony structure of the anus of the second female for deterring forwardly loose linear travel of the insertion shaft (1) in the vagina of the first female and by the vaginal-anchor hook (11) being buttressed by the bony structure of the symphysis pubis of the second female for deterring rearwardly loose linear travel of the insertion shaft (1) in the vagina of the first female.

The attachment base (2) can be formed for deterring unwantedly loose lateral travel of the vaginal anchor (5) in the vagina of the second female by the double anchoring with the anal-anchor (4) being buttressed by the aft bony structure of the anus of the second female and the vaginal-anchor hook (11) being buttressed by the bony structure of the symphysis pubis of the second female.

Preferably, the double-anchor strapless dildo is rigid, washable, sanitizable and optionally smooth or softly rough-surfaced.

A new and useful double-anchor strapless dildo having been described, all such foreseeable modifications, adaptations, substitutions of equivalents, mathematical possibilities of combinations of parts, pluralities of parts, applications and forms thereof as described by the following claims and not precluded by prior art are included in this invention.

1-20. (canceled)

21. A double-anchor strapless dildo comprising:
an insertion shaft (1) extended intermediate an attachment base (2) and an insertion end (3);
the insertion shaft (1) having length and breadth for gratifying insertion into a vagina of a first female;
the attachment base (2) having an anal anchor (4) and a vaginal anchor (5);
the vaginal anchor (5) having length and breadth for gratifying insertion into a vagina of a second female;
the anal anchor (4) being extended predeterminedly from an attachment side of the attachment base (2); and
the vaginal anchor (5) being extended predeterminedly from the attachment side of the attachment base (2) intermediate the anal anchor (4) and the insertion shaft (1).

22. The double-anchor strapless dildo of claim 21 wherein:
the anal anchor (4) has breadth for ease of insertion into an anus of the second female and form for deterring escape from the anus of the second female.

23. The double-anchor strapless dildo of claim 21 wherein:
the vaginal anchor (5) has breadth for ease of insertion into the vagina of the second female and for deterring escape from the vagina of the second female.

24. The double-anchor strapless dildo of claim 23 wherein:
the anal anchor (4) has breadth for ease of insertion into an anus of the second female and form for deterring escape from the anus of the second female.

25. The double-anchor strapless dildo of claim 21 wherein:
the anal anchor (4) includes a conically circular point (6) having a circular base (7) extended radially outward from an anal-anchor protrusion (8) extended from the attachment side of the attachment base (2);
the conically circular point (6) is blunted and elongated predeterminedly for ease of entry into and gentle spread of the anal orifice of the second female;
the conically circular base (7) includes a rounded edge for hooking bluntly onto an inside of an entry wall of the anal orifice of the second female; and
the anal-anchor protrusion (8) includes a length that is proximate a thickness of the entry wall of the anal orifice of the second female.

26. The double-anchor strapless dildo of claim 21 wherein:
the vaginal anchor (5) includes a vaginal-anchor shaft (9) having length, breadth and shape for gratifying insertion into the vagina of the second female.

27. The double-anchor strapless dildo of claim 26 wherein:
the vaginal-anchor shaft (9) includes a predetermined vaginal-anchor head (10) formed and structured proximate a terminal end (14) for stimulating entry into and linear positioning within the vagina of the second female.

28. The double-anchor strapless dildo of claim 26 wherein:
the vaginal anchor (5) includes a predetermined vaginal-anchor hook (11) proximate a base end of the vaginal-anchor shaft (9); and
the vaginal-anchor hook (11) is formed and positioned for engaging forward walls of the vagina and for engaging bony structure of the symphysis pubis of the second female.

29. The double-anchor strapless dildo of claim 21 wherein:
the vaginal anchor (5) includes a predetermined vaginal-anchor hook (11) proximate a base end of the vaginal-anchor shaft (9); and
the vaginal-anchor hook (11) is formed and positioned for engaging erogenous entry zones of the vagina predeterminedly and for hooking engagement of bony structure of the symphysis pubis of the second female predeterminedly.

30. The double-anchor strapless dildo of claim 21 wherein:
the insertion shaft (1) includes at least one predetermined insertion-shaft head (12) formed and positioned on the insertion shaft (1) for engaging predetermined erogenous zones of the vagina of the first female.

31. The double-anchor strapless dildo of claim 21 wherein:
the insertion end (3) of the insertion shaft (1) includes the predetermined insertion-shaft head (12) formed and positioned on the insertion shaft (1) for engaging erogenous zones of the internal end of the vagina of the first female.

32. The double-anchor strapless dildo of claim 21 wherein:
the insertion shaft (1), the anal anchor (4) and the vaginal anchor (5) are predeterminedly rigid and washable.

33. The double-anchor strapless dildo of claim 21 wherein:
the insertion shaft (1) is structured for deterrence of lateral pivot by double anchoring with the anal anchor (4) and the vaginal anchor (5).

34. The double-anchor strapless dildo of claim 21 wherein:
the attachment base (2) is formed for deterring unwantedly loose linear travel of the insertion shaft (1) in the vagina of the first female by double anchoring with the anal anchor (4) being buttressed by aft bony structure of the anus of the second female for deterring forwardly loose linear travel of the insertion shaft (1) in the vagina of the first female and by the vaginal-anchor hook (11) being buttressed by the bony structure of the symphysis pubis of the second female for deterring rearwardly loose linear travel of the insertion shaft (1) in the vagina of the first female.

35. The double-anchor strapless dildo of claim 21 wherein:
the attachment base (2) is formed for deterring unwantedly loose lateral travel of the vaginal anchor (5) in the vagina of the second female by the double anchoring with the anal-anchor (4) being buttressed by the aft bony structure of the anus of the second female and the vaginal-anchor hook (11) being buttressed by the bony structure of the symphysis pubis of the second female.

36. A double-anchor strapless dildo comprising:
the insertion shaft (1) formed for linear travel in the vagina of the first female with the anal anchor (4) formed for anal-anchoring insertion in the anal orifice of the second female combined with the vaginal anchor (5) formed for vaginal-anchoring insertion in the vagina of the second female.

37. The double-anchor strapless dildo of claim 36 wherein:
the attachment base (2) is formed for deterring unwantedly loose linear travel of the insertion shaft (1) in the vagina of the first female by double anchoring with the anal anchor (4) being buttressed by aft bony structure of the anus of the second female for deterring forwardly loose linear travel in the vagina of the first female and by the vaginal-anchor hook (11) being buttressed by the bony structure of the symphysis pubis of the second female for deterring rearwardly loose linear travel in the vagina of the first female.

38. A double-anchor strapless dildo comprising:
the insertion shaft (1) formed for linear travel in the vagina of the first female with the anal anchor (4) formed for anal-anchoring insertion in the anal orifice of the first female combined with the vaginal anchor (5) formed for vaginal-anchoring insertion in the vagina of the second female;
the attachment base (2) being formed for deterring unwantedly loose lateral travel of the vaginal anchor (5) in the vagina of the second female by the double anchoring with the anal-anchor (4) being buttressed by the aft bony structure of the anus of the second female and the vaginal-anchor hook (11) being buttressed by the bony structure of the symphysis pubis of the second female;
the anal anchor (4) having breadth for ease of insertion into an anus of the second female and having form for deterring escape from the anus of the second female;
the vaginal anchor (5) having breadth for ease of insertion into the vagina of the second female and for deterring escape from the vagina of the second female;
the anal anchor (4) having the conically circular point (6) with the circular base (7) extended radially outward from an anal-anchor protrusion (8) extended from the attachment side of the attachment base (2);
the conically circular point (6) being blunted and elongated predeterminedly for ease of entry into and gentle rate of spread of the anal orifice of the second female;
the conically circular base (7) having the rounded edge for hooking bluntly onto an inside of an entry wall of the anal orifice of the second female;
the anal-anchor protrusion (8) having the length that is proximate a thickness of the entry wall of the anal orifice of the second female;
the vaginal anchor (5) having the vaginal-anchor shaft (9) with length, breadth and shape for gratifying insertion into the vagina of the second female;
the vaginal-anchor shaft (9) having the predetermined vaginal-anchor head (10) formed and structured proximate the terminal end (14) for stimulating entry and linear positioning in the vagina of the second female;
the vaginal anchor (5) having the predetermined vaginal-anchor hook (11) proximate the base end of the vaginal-anchor shaft (9);
the vaginal-anchor hook (11) being formed and positioned for engaging forward walls of the vagina and for engaging bony structure of the symphysis pubis of the second female;
the vaginal-anchor hook (11) being formed and positioned for engaging erogenous entry zones of the vagina of the second female predeterminedly;
the insertion shaft (1) having at least one predetermined insertion-shaft head (12) formed and positioned on the insertion shaft (1) for engaging predetermined erogenous zones of the vagina of the first female;
the insertion end (3) of the insertion shaft (1) having the predetermined insertion-shaft head (12) formed and positioned on the insertion shaft (1) for engaging erogenous zones of the internal end of the vagina of the first female; and
the insertion shaft (1), the anal anchor (4) and the vaginal anchor (5) being predeterminedly rigid and washable.

39. The double-anchor strapless dildo of claim 21 wherein:
the double-anchor strapless dildo is rigid, washable, sanitizable and smooth-surfaced predeterminedly.

40. The double-anchor strapless dildo of claim 36 wherein:
the double-anchor strapless dildo is rigid, washable, sanitizable and softly rough-surfaced predeterminedly.
